(19) United States
(12) Patent Application Publication
(10) Pub. No.: US 2006/0190088 A1
(43) Pub. Date: Aug. 24, 2006

(54) Prosthesis for correction of flatfoot deformity

(76) Inventors: Brent G. Parks, West Friendship, MD (US); Lew C. Schon, Baltimore, MD (US); Christopher P. Chiodo, Walpole, MA (US)

Correspondence Address: DICKSTEIN SHAPIRO MORIN & OSHINSKY LLP, 2101 L Street, NW, Washington, DC, 20037, US

(21) Appl. No.: 11/359,469

(22) Filed: Feb. 23, 2006

Related U.S. Application Data
(60) Provisional application No. 60/655,712, filed on Feb. 23, 2005.
(60) Provisional application No. 60/665,712, filed on Mar. 28, 2005.

(51) Int. Cl. A61F 2/42

(52) U.S. Cl. 623/21.11; 606/73; 623/23.44

(57) ABSTRACT

An internal human prosthesis used in surgical procedures to treat flatfoot deformities, including pediatric, adult congenital, and adult acquired deformities. The internal prosthesis is inserted between the human talus and calcaneus bones. In a preferred embodiment, the prosthesis has a truncated conical shape along its long axis and a central bore through the center of its long axis. The base of the prosthesis includes a concave depression with a polygonal recess in its center.

### CROSS-REFERENCE TO RELATED APPLICATIONS

This application claims priority from U.S. Provisional Application No. 60/665,712, filed Feb. 23, 2005, the entire disclosure of which is incorporated by reference herein.

### BACKGROUND OF THE INVENTION

Flatfoot deformity, also known as pes planus, is a result of a loss of the normal medial longitudinal arch. As a result, the calcaneus bone may lie in valgus and external rotation relative to the talus. This deformity occurs in both children and adults and, in some cases, may limit normal function. Flatfoot deformity may cause pain at the foot or ankle, pain while walking or standing, or lower back and knee pain.

Many times, the symptoms of flatfoot deformity may be treated using non-surgical solutions such as orthotics, anti-inflammatory medications or ice and rest. However, sometimes the symptoms are too severe or remain even with these more conservative treatments. One option for surgical treatment of flatfoot deformity is the insertion of a prosthesis into to sinus tarsi opening. The prosthesis restores the correct arch of the foot by preventing the displacement of the talus bone and preventing pronation of the foot. Typically, this implant has been in the form of a conical or rounded shape internal prosthesis.

Despite the extensive development of internal human prostheses, they continue to exhibit certain disadvantages. For example, they are difficult to insert and remove by virtue of the shape and design of their base (which typically lack features for ease of removal, although the base is the end positioned closest to the surface after insertion). Thus, there exists a continuing need for the development of new and improved prostheses for the treatment of foot deformities such as flatfoot.

### SUMMARY OF THE INVENTION

Recognizing the need for the development of an improved implantable prosthesis for the correction of a human flatfoot deformity, the present invention is generally directed to alleviate the disadvantages of prior art devices as well as to provide other advantages over the prior art.

The present invention provides an internal human prosthesis for use in surgical procedures to treat flatfoot deformities, including pediatric, adult congenital, and adult acquired deformities. The internal prosthesis, designed for correction of a flatfoot deformity, is inserted between the human talus and calcaneus bones. The prosthesis preferably has a truncated conical shape along its long axis with a convex or tapered tip and a central bore through the center of its long axis. Advantageously, the base of the prosthesis includes a concave depression with a recess in its center.

When the prosthesis is inserted between the talus and calcaneus bones, the alignment of these bones is improved by virtue of the shape of the prosthesis. More particularly, the neck of the talus bone is elevated and the calcaneus is inverted. Once fully inserted, the prosthesis also prevents excessive eversion of the calcaneus bone.

### BRIEF DESCRIPTION OF THE DRAWINGS

The foregoing and other advantages and features of the invention will be more readily understood from the following detailed description of the invention provided below with reference to the accompanying drawings, in which:

FIG. 1A illustrates a bottom perspective view of a first embodiment of the present invention having a truncated pyramidal polyhedral shape;

FIG. 1B illustrates a side view of the first embodiment of the present invention;

FIG. 1C illustrates a bottom plan view of the first embodiment of the present invention;

FIG. 1D illustrates a cross-sectional view along line A of FIG. 1C;

FIG. 2A illustrates a bottom perspective view of the first embodiment of the present invention;

FIG. 2B illustrates a bottom perspective view of a second embodiment of the present invention, having a four-sided truncated pyramidal polyhedral shape;

FIGS. 2C-2E illustrate various views of a preferred embodiment of the present invention in the shape of a truncated cone;

FIG. 3A illustrates one set of typical dimensions for the prosthesis of the present invention; and

FIG. 3B illustrates a second set of typical dimensions for the prosthesis of the present invention.

### DETAILED DESCRIPTION OF THE INVENTION

In the following detailed description, reference is made to the accompanying drawings, which form a part hereof and show by way of illustration specific embodiments in which the invention may be practiced. These embodiments are described in sufficient detail to enable those skilled in the art to practice the invention, and it is to be understood that other embodiments may be utilized, and that changes may be made without departing from the spirit and scope of the present invention.

The present invention provides an internal prosthesis for correcting a flatfoot deformity that is simple to construct and use with low manufacturing costs, is more stable than other prostheses by virtue of its shape, and facilitates insertion, positioning, and removal by virtue of the unique shape of the concave base of the prosthesis with a polyhedral recess at its center.

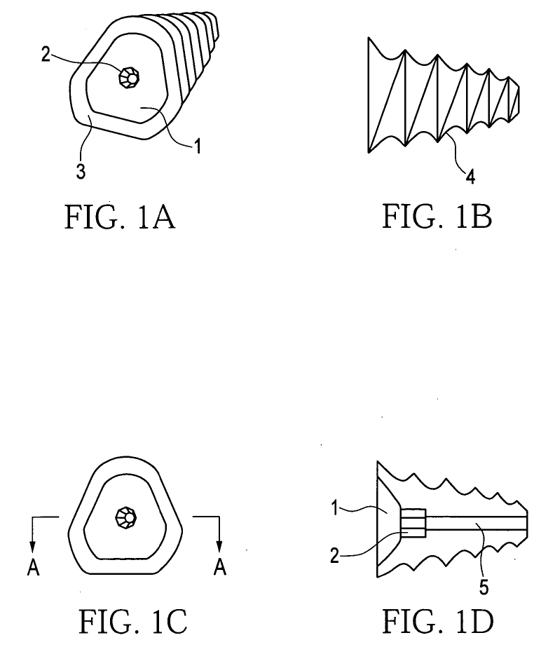

FIGS. 1A-1D illustrate a first embodiment of the prosthesis 10 in a bottom perspective view, a side view, a bottom plan view, and a cross-sectional view, respectively. The prosthesis 10 has a truncated pyramidal polyhedral shape along its long axis 8 with a base 6 that is comprised of or has within it a concave depression 1 with a polyhedral recess 2 in its center. The opposite end 7 of the prosthesis 10 is truncated and convex or tapered.

The surface of the concave depression 1 can either be smooth or include ridges to help guide a guide wire or drive toward the cannulation 5. The depth of the concave depression 1 may vary as well. The concave depression 1 may comprise the entire base 6 or may cover only a portion of the base 6. When the concave depression 1 comprises the entire base 6, lip 3 is not present. When the concave depression 1 comprises only a portion of the base 6, then lip 3 is present around the concave depression 1. The lip width may vary depending on the design considerations like the materials used for fabrication.

The base 6 of the prosthesis 10 preferably includes a polyhedral recess 2 in its center. This recess 2 is configured to receive a driving instrument which is used during insertion and/or removal of the prosthesis 10. The recess 2 may have a different number of sides, and be a shape such as a square, hexagonal, octagonal or a Trox drive design. In the preferred embodiment, the recess 2 is shaped to fit any standard driver, such as a 3.5 mm hex driver. Additionally, the recess 2 may be tapered along the long axis 8 of the prosthesis 10 and the depth and diameter of the recess 2 may vary. One skilled in the art would appreciate that any shape, depth and diameter may be used for recess 2 as long as the recess 2 corresponds with the driver being used for insertion and/or removal of the prosthesis 10. Cannulation or central bore 5 is included along the long axis 8 of the prosthesis 10 in order to facilitate placement over a guide wire during insertion of the prosthesis.

The prosthesis 10 preferably includes threads 4 along its body. The threads 4 are configured for ease of insertion and retention of the prosthesis 10. The thread pitch and depth are matters of design choice, but will be such that the device is easily inserted and provides a predetermined resistance to being forced or “popped” back out of the intended location of the sinus tarsi during normal use. The thread pitch may be uniform or variable. Additionally, the threads 4 can be either continuous or interrupted (i.e., not one single continuous thread). Optimal designs will provide improved purchase with the sinus tarsi and better interdigitation of soft tissues. Alternatively, the threads may be replaced by ridges, protrusions, or slots/perforations.

In operation, the prosthesis 10 may be inserted over a guide wire or rod that is first placed in the region of the sinus tarsi, between the talus and calcaneus bones. The guide wire or rod is inserted between the neck of the talus bone and the anterior body of the calcaneus bone from lateral to medial using fluoroscopic guidance if necessary. The central bore 5 of the prosthesis 10 is then inserted over the wire using an inserter/removal device comprised of a handle, shaft, and polygonal head. The inserter/removal device may be any convenient wrench-type device. The polygonal head of the inserting device fits into a corresponding and similarly shaped recess 2 located in the center of the concave depression 1 on the base 6 of the prosthesis 10.

If the prosthesis 10 needs to be removed, removal is facilitated by virtue of the unique shape of the base 6 of the prosthesis with the polyhedral recess 2 at its center. More particularly, the head of the inserter/removal device can be mated with the polyhedral recess 2 more readily by virtue of the concave nature of the depression 1 within or comprising the base 6 of the prosthesis 10.

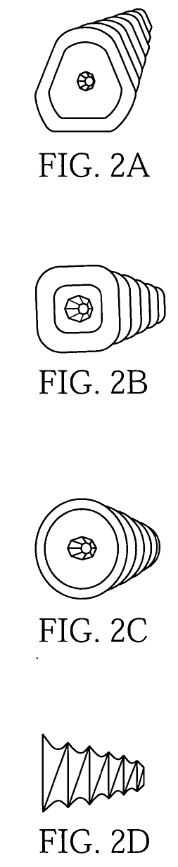

In the preferred embodiment, the shape of the prosthesis 10 is a truncated cone, as shown in FIGS. 2C, 2D and 2E. The prosthesis may also have a pyramidal polyhedral shape, as shown in FIGS. 2A and 2B, which allows better fill of the cavity in which the prosthesis 10 sits, and resists backing-out of the prosthesis 10. Tapered and pyramidal shapes require fewer sizes to accommodate anthropometric variable among patients.

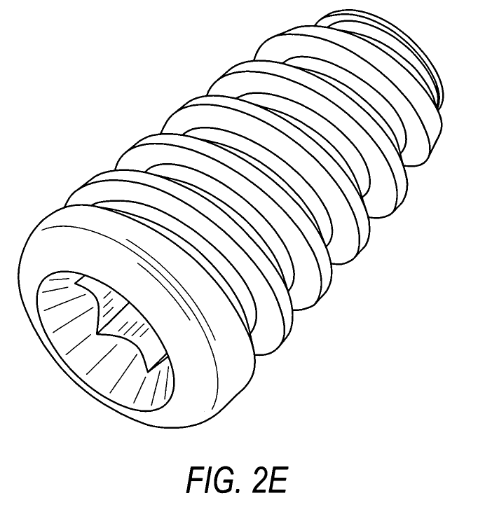

The size of the prosthesis 10 may vary to allow for accommodation of different patients. FIGS. 3A and 3B illustrate prostheses of two different sizes that would cover the majority of cases. The larger size in FIG. 3A would be appropriate for adults. The smaller size in FIG. 3B would be appropriate for children or small adults. One skilled in the are will readily appreciate how to select an appropriately shaped and sized prosthesis for a particular patient. Additionally, he/she would be able to readily determine how deep and at what orientation to insert the prosthesis, based on the alignment desired between the talus and calcaneus bones after insertion.

The prosthesis 10 may be composed of a metal, ceramic, polymer, bioresorbable, or biological material, including autograft, allograft, xenograft, or engineered tissue material. The material may also be modified with regard to radio-opacity in order to facilitate insertion, removal, and positioning.

Of course, one skilled in the art will appreciate how a variety of alternatives are possible for the individual elements, and their arrangement, described above, while still falling within the spirit of the invention. While the above describes several embodiments of the invention used primarily in connection with treating flatfoot disorder, those skilled in the art will appreciate that there are a number of alternatives, based on system design choices and choice of protocol options, and extensions that still fall within the spirit of the invention. Thus, it is to be understood that the invention is not limited to the embodiments described above, and that in light of the present disclosure, various other embodiments and applications should be apparent to persons skilled in the art. Accordingly, it is intended that the invention not be limited to the specific illustrative embodiments.

What is claimed as new and desired to be protected by Letters Patent of the United States is:

1. A prosthesis for correcting a human flatfoot deformity comprising:
a body configured for insertion between the neck region of the talus bone and the calcaneus bone in a region of the sinus tarsi, wherein the body is oriented such that, when inserted, a long axis of the body approximately parallels a medial-lateral human anatomical axis, and wherein the body has a base end provided with a concave depression and an end opposite the base, and wherein the body is tapered along its long axis such that a cross-sectional area of the end of the body opposite the base is smaller than a cross-sectional area of the body at the base.

2. The prosthesis of claim 1, wherein the body is formed in the shape of a truncated cone.

3. The prosthesis of claim 1, wherein the body is formed in the shape of a truncated pyramidal polyhedral.

4. The prosthesis of claim 1, wherein the depression has a cross-sectional area which is smaller or equal to a cross-sectional area of the base.

5. The prosthesis of claim 4, wherein the concave depression has a variable depth along the long axis of the body.

6. The prosthesis of claim 5, wherein the concave depression may comprise at least one groove or ridge, thereby facilitating insertion and/or removal of the prosthesis.

7. The prosthesis of claim 6, wherein the concave depression has a uniform or tapered polyhedral recess, the recess being contiguous with a center of the concave depression and the center of the recess being oriented along the long axis of the body, further facilitating the insertion and/or removal of the prosthesis.

8. The prosthesis of claim 1, wherein the end opposite the base is truncated.

9. The prosthesis of claim 1, wherein the end opposite the base is convex or tapered.

10. The prosthesis of claim 1, further comprising an axial bore oriented along the long axis of the body.

11. The prosthesis of claim 1, further comprising threads along at least one portion of a surface of the prosthesis between the base and the end opposite the base, thereby facilitating soft-tissue apposition, interdigitation, and adherence.

12. The prosthesis of claim 11, wherein the threads are continuous.

13. The prosthesis of claim 11, wherein the threads are interrupted.

14. The prosthesis of claim 11, wherein the threads have variable pitch.

15. The prosthesis of claim 11, wherein the threads have uniform pitch.

16. The prosthesis of claim 11, wherein the threads have variable width and height.

17. The prosthesis of claim 11, wherein the threads have uniform width and height.

18. The prosthesis of claim 11, wherein the threads have one of a group of variable or uniform shapes including substantially v-shaped, right triangle, and sinusoidal.

19. The prosthesis of claim 1, comprising at least one of metal, ceramic, polymer, bioresorbable, or biological material, including autograft, allograft, xenograft, or engineered tissue material.

20. The prosthesis of claim 19, comprising a material that may be modified with regard to radio-opacity, thereby facilitating insertion, removal, and positioning.
